(19) United States
(12) Patent Application Publication
(10) Pub. No.: US 2005/0186674 A1
(43) Pub. Date: Aug. 25, 2005

(54) Patterning substrate and cell culture substrate

(76) Inventors: Hideyuki Miyake, Tokyo (JP); Hideshi Hattori, Tokyo (JP); Hironori Kobayashi, Tokyo (JP)

Correspondence Address: SEYFARTH SHAW, 55 EAST MONROE STREET, SUITE 4200, CHICAGO, IL, 60603-5803, US

(21) Appl. No.: 10/986,641

(22) Filed: Nov. 12, 2004

(30) Foreign Application Priority Data
Nov. 14, 2003 (JP) 2003-385020

(51) Int. Cl. C12N 5/02; H01L 21/00

(52) U.S. Cl. 435/404; 438/1

(57) ABSTRACT

The present invention intends primarily to provide such as a cell culture patterning substrate that is used to adhere a cell in a highly precise pattern on a base material to culture and a cell culture substrate on which a cell is adhered in a high precision pattern. In order to achieve the above-mentioned object, the present invention provides a patterning substrate having a base material and a cell culture patterning layer that is formed on the base material and has at least a photocatalyst and a cell adhesive material that has the adhesiveness with the cell and is decomposed or modified owing to an action of the photocatalyst in combination with energy irradiation.

### BACKGROUND OF THE INVENTION

1. Field of the Invention

The present invention relates to a cell culture patterning substrate capable of adhering cells in a highly precise pattern, a patterning substrate used for forming the cell culture patterning substrate, a coating liquid for patterning substrate used for forming the patterning substrate, and a cell culture substrate on which cells are adhered in a highly precise pattern.

2. Description of the Related Art

At present, cell cultures of various animals and plants are performed, and also new cell culture methods are in development. The technologies of the cell culture are utilized, such as, to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method of arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in Japanese Patent Application Laid-Open (JP-A) No. 2-245181, an electric charge-retaining medium on which an electrostatic pattern is formed is applied to culture cells for the purpose of proliferating nerve cells in a form of circuit, and the like. Furthermore, JP-A No. 3-7576 tries to arrange cultured cells on a surface on which a cell non-adhesive or cell adhesive photosensitive hydrophilic polymer has been patterned by a photolithography method.

Furthermore, JP-A No. 5-176753 discloses a cell culture base material on which a substance such as collagen and the like affecting on the adhesion ratio and form of cells is patterned, and a method of producing this base material by a photolithography method. By culturing cells on such a base material, a larger amount of cells can be adhered on a surface on which collagen or the like is patterned, to realize patterning of cells.

However, such patterning of cell culture regions may be required to be highly precise depending on applications. In the case of conducting patterning by a photolithography method using a photosensitive material as described above, a highly precise pattern can be obtained; however, a cell adhesive material is required to have photosensitivity, and it is difficult in many cases to conduct chemical modification to impart such photosensitivity to, for instance, biopolymers and the like; thereby leading to extremely narrow width in selectivity of cell adhesive materials, problematically. Furthermore, in a photolithography method using a photo resist, it is necessary to use a liquid developer and the like, and these can affect adversely in culturing cells in some cases.

Furthermore, as a method of forming a highly precise pattern of a cell adhesive material, a Micro Contact Printing method is proposed by George M. Whitesides, Harvard University (for example, U.S. Pat. Nos. 5,512,131 and 5,900,160, JP-A Nos. 9-240125 and 10-12545 etc.). However, there is a problem in that it is difficult to industrially produce a cell culture base material having a pattern of a cell adhesive material using this method.

### SUMMARY OF THE INVENTION

In this connection, it is desired to provide such as a cell culture patterning substrate that is used to cause cells to adhere in highly precise pattern onto a base material to culture, and a cell culture substrate to which cells are adhered in highly precise pattern.

The present invention provides a patterning substrate having a base material; and a cell culture patterning layer that is formed on the base material and contains at least a photocatalyst and a cell adhesive material that has an adhesiveness with the cells and can be decomposed or modified owing to an action of a photocatalyst in combination with energy irradiation.

In the present invention, since the cell culture patterning layer contains the photocatalyst and the cell adhesive material, by irradiating energy, owing to an action of the photocatalyst in combination with the energy irradiation, regions where the cell adhesive material is decomposed or modified can be formed. Therefore, according to the present invention, without requiring a particular device or a complicated process, a patterning substrate on which a region containing the cell adhesive material that is excellent in the adhesiveness with the cells and a region where the cell adhesive material is decomposed or modified and the adhesiveness with the cells is low can be easily formed can be obtained.

In the above-mentioned invention, on the base material, a light-shielding portion can be formed in a pattern. In this case, when energy is irradiated from a base material side, energy can be irradiated only on a cell culture patterning layer in a region where the light-shielding portion is not formed; accordingly, a patterning substrate on which a pattern where the cell adhesive material remains in a pattern according to which the light-shielding portion is formed can be formed can be obtained.

In the present invention, the cell culture patterning layer preferably contains a binder. Thereby, the characteristics such as imparting the strength to the cell culture patterning layer and lowering the adhesiveness with the cells in a region irradiated by energy can be imparted.

The present invention provides a cell culture patterning substrate having a base material and a cell culture pattern layer formed on the base material, wherein the cell culture pattern layer having a cell adhesion portion that contains at least a photocatalyst and a cell adhesive material that has an adhesiveness with the cells and can be decomposed or modified owing to an action of the photocatalyst in combination with energy irradiation; and a cell non-adhesion portion that contains at least a photocatalyst and where the cell adhesive material is decomposed or modified.

According to the present invention, since the cell culture pattern layer has the cell adhesion portion and the cell non-adhesion portion, cells can be readily adhered only onto the cell adhesion portion. Thereby, for instance, even when cells are coated over an entire surface of the cell culture pattern layer, a cell culture patterning substrate on which only on the cell adhesion portion, the cells can be adhered with high precision can be obtained.

In the present invention, one in which on the base material, a light-shielding portion is formed with the same pattern as that of the cell adhesion portion can be prepared. Thereby, when a cell culture patterning substrate is formed, by irradiating energy from the base material side, the cell adhesive material in a region other than a region where the light-shielding portion is formed can be decomposed or modified, and thereby a cell non-adhesion portion can be readily formed. Furthermore, when cells are adhered to the cell adhesion portion on the cell culture patterning substrate according to the present invention to form a cell culture substrate, by irradiating energy from the base material side, the cells present in the cell non-adhesion portion can be readily removed owing to an action of a photocatalyst in combination with energy irradiation, and thereby the cells can be held in highly precise pattern.

In the above-mentioned invention, the cell culture pattern layer preferably contains a binder. Thereby, the characteristics such as the strength of the cell culture pattern layer being improved and the cells being inhibited further from adhering onto the cell non-adhesion portion can be imparted.

The present invention also provides a cell culture substrate in which cells are adhered onto the cell adhesion portion in any one of the above-mentioned cell culture patterning substrates.

According to the present invention, cells do not adhere onto the cell non-adhesion portion of the cell culture patterning substrate and adhere only onto the cell adhesion portion; accordingly, without applying a complicated process or using a processing liquid and the like that adversely affects on the cells, a cell culture substrate on which cells are readily adhered in highly precise pattern can be prepared.

The present invention provides a coating liquid for patterning substrate that contains at least a photocatalyst and a cell adhesive material that has an adhesiveness with the cells and can be decomposed or modified owing to an action of the photocatalyst in combination with energy irradiation.

According to the present invention, owing to the inclusion of the photocatalyst and the cell adhesive material, when the coating liquid for patterning substrate is coated on the base material, for instance, followed by irradiating energy in a pattern, owing to an action of the photocatalyst in combination with energy irradiation, a region where the cell adhesive material is decomposed or modified and a region of which adhesiveness with the cells are excellent owing to the cell adhesive material can be readily formed.

In the present invention, the coating liquid for patterning substrate preferably contains a binder. Thereby, when the coating liquid for patterning substrate is coated to form a layer, a layer can, for instance, be readily formed and the strength of the layer can be improved. Furthermore, after the layer is formed, the adhesiveness with the cells in the region where energy is irradiated can be controlled and the like.

The present invention is a method of manufacturing a cell culture substrate having a base material; a cell culture pattern layer that is formed on the base material and has a cell adhesion portion that contains at least a photocatalyst and a cell adhesive material that has an adhesiveness with cells and can be decomposed or modified owing to an action of the photocatalyst in combination with energy irradiation and a cell non-adhesion portion that contains at least a photocatalyst and where the cell adhesive material is decomposed or modified; and cells adhered on the cell adhesion portion, the method comprising a cell maintaining process for irradiating energy on the cell non-adhesion portion to maintain a pattern of the cell adhered onto the cell adhesion portion, after a cell adhering process for adhering a cell on the cell adhesion portion is carried out.

According to the above-mentioned invention, owing to the cell adhering process, after the cells are adhered on the cell adhesion portion, owing to the cell maintaining process, by irradiating energy on the cell non-adhesion portion on the cell culture substrate, the cells and the like adhered onto the cell non-adhesion portion can be removed, and thereby a cell culture substrate on which only on a cell adhesion portion cells are adhered in highly precise pattern can be prepared.

### BRIEF DESCRIPTION OF THE DRAWINGS

FIG. 1 is a schematic sectional view showing one example of the patterning substrate in the present invention.

FIG. 2 is a schematic sectional view showing another example of the patterning substrate in the present invention.

FIG. 3 is a schematic sectional view showing one example of the cell culture patterning substrate in the present invention.

FIG. 4 is a schematic sectional view showing another example of the cell culture patterning substrate in the present invention.

FIGS. 5A to 5C are process charts showing one example of the methods of forming a cell culture pattern layer in a cell culture patterning substrate in the present invention.

FIG. 6 is a schematic sectional view showing one example of the cell culture substrate in the present invention.

FIG. 7 is a schematic sectional view showing one example of the energy irradiating process in the method of manufacturing a cell culture substrate in the present invention.

FIG. 8 is a schematic sectional view showing another example of the energy irradiating process in the method of manufacturing a cell culture substrate in the present invention.

### DESCRIPTION OF THE PREFERRED EMBODIMENTS

The present invention relates to such as a cell culture patterning substrate on which cells can be adhered in highly precise pattern, a patterning substrate that is used to form the cell culture patterning substrate, a coating liquid for patterning substrate that is used to form the patterning substrate, and a cell culture substrate on which cells are adhered in highly precise pattern. Hereinafter, these will be explained below separately.

A. Coating Liquid for Patterning Substrate

First, a coating liquid for patterning substrate will be explained in detail. A coating liquid for patterning substrate according to the present invention contains at least a photocatalyst and a cell adhesive material that has the adhesiveness with cells and can be decomposed or modified owing to an action of the photocatalyst in combination with energy irradiation.

In the coating liquid for patterning substrate according to the present invention, the cell adhesive material is contained; accordingly, when the coating liquid for patterning substrate is applied on a base material to form a layer, owing to the cell adhesive material, the adhesiveness with the cells can be made excellent. On the other hand, when energy is irradiated on the layer, owing to an action of the photocatalyst contained in the coating liquid for patterning substrate, the cell adhesive material can be decomposed or modified, and thereby one in which the adhesiveness with the cells is lowered can be prepared. Accordingly, when the coating liquid for patterning substrate according to the present invention is used to form a layer, followed by irradiating energy in a pattern, one having a region excellent in the adhesiveness with the cells including the cell adhesive material and a region where the cell adhesive material is decomposed or modified and thereby the adhesiveness with the cells is lowered can be formed.

Hereinafter, the respective materials that are used in the coating liquid for patterning substrate according to the present invention will be explained in detail.

1. Photocatalyst

At the beginning, a photocatalyst used in a coating liquid for patterning substrate according to the present invention will be explained. The photocatalyst used in the present invention, as far as it can decompose or modify a cell adhesive material described below owing to an action of the photocatalyst in combination with energy irradiation, is not particularly restricted.

Though the action mechanism of a photocatalyst typical in titanium oxide described below is not necessarily clear, it can be considered that a carrier generated by irradiation of light directly reacts with a nearby compound or, owing to an active oxygen species generated under the presence of oxygen, water, a chemical structure of an organic material is caused to change. In the present invention, it is considered that this carrier affects on a cell adhesive material described below.

As the photocatalyst that can be used in the present invention, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present invention, in particular, titaniumdioxide, owing to a large band gap, chemical stability, non-toxicity, and easy availability, can be preferably used. There are two types of titanium dioxide, anatase type and rutile type, and both can be used in the present invention; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-K01, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

A content of the photocatalyst in the coating liquid for patterning substrate according to the present invention can be set in the range of 5 to 95% by weight, preferably of 10 to 60% by weight, and more preferably of 20 to 40% by weight. Thereby, when the coating liquid for patterning substrate is coated to form a layer, a cell adhesive material in a region where energy is irradiated can be decomposed or modified.

The photocatalyst used in the present invention, owing to, for instance, high hydrophilicity thereof and the like, is preferably low in the adhesiveness with cells. Thereby, a region where the photocatalyst is exposed owing to such as the decomposition of a cell adhesive material described below can be used as a region low in the adhesiveness with the cells.

2. Cell Adhesive Material

In the next place, a cell adhesive material used in the coating liquid for patterning substrate according to the present invention will be explained. The cell adhesive material used in the coating liquid for patterning substrate according to the present invention, as far as it has the adhesiveness with cells and can be decomposed or modified owing to an action of a photocatalyst in combination with energy irradiation, is not particularly restricted to the kind and the like. Here, “having the adhesiveness with cells” means being excellent in the adhesiveness with the cells, and, for instance, when the adhesiveness with cells is different depending on the kind of cells, means to be excellent in the adhesiveness with target cells.

The cell adhesive material used in the present invention has such adhesiveness with cells and can be decomposed or modified owing to an action of a photocatalyst in combination with energy irradiation such as to lose the adhesiveness with the cells or change to one that has the cell adhesion inhibiting properties that inhibit to adhere to cells.

As such materials having the adhesiveness with the cells, there are two kinds, one being materials having the adhesiveness with cells owing to physicochemical characteristics and the other being materials having the adhesiveness with cells owing to biochemical characteristics.

As physicochemical factors that determine the adhesiveness with cells of materials having the adhesiveness with cells owing to the physicochemical characteristics, the surface free energy, the electrostatic interaction and the like can be cited. For instance, in the case of the adhesiveness with cells being determined by the surface free energy of the material, when the material has the surface free energy in a predetermined range, the adhesiveness between the cells and the material becomes excellent, and when it deviates from the above range the adhesiveness between the cells and material decreases. As such changes of the adhesiveness of cells due to the surface free energy, experimental results such as shown in Data, for instance, CMC Publishing Co., Ltd. “Biomaterial no Saisentan”, Yoshito IKADA (editor), p. 109, lower part are known. As materials having the adhesiveness with cells owing to such a factor, for instance, hydrophilic polystyrene, poly (N-isopropyl acrylamide) and the like can be cited. When such a material is used, owing to an action of a photocatalyst in combination with energy irradiation, for instance, a functional group on a surface of the material is substituted, decomposed or the like to cause a change in the surface free energy, resulting in one that does not have the adhesiveness with cells or one that has the cell adhesion inhibiting properties.

When the adhesiveness between cells and a material is determined owing to the electrostatic interaction or the like, for instance, the adhesiveness with cells can be determined owing to an amount of positive electric charges and the like that the material has. As materials having the adhesiveness with cells owing to such electrostatic interaction, basic polymers such as polylysine, basic compounds such as aminopropyltriethoxysilane, N-(2-aminoethyl)-3-aminopropyltrimethoxysilane and condensates and the like including these can be cited. When such materials are used, owing to an action of a photocatalyst in combination with energy irradiation, the above-mentioned materials are decomposed or modified, thereby, for instance, an amount of positive electric charges present on a surface can be altered, resulting in one that does not have the adhesiveness with the cells or one that has the cell adhesion inhibiting properties.

As materials having the adhesiveness with cells owing to the biological characteristics, ones that are excellent in the adhesiveness with particular cells or ones that are excellent in the adhesiveness with many cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited. When such materials are used, owing to an action of a photocatalyst in combination with energy irradiation, for instance, a structure of the material is partially destroyed, a principal chain is destroyed or the like, resulting in one that does not have the adhesiveness with cells or one that has the cell adhesion inhibiting properties.

Such a cell adhesive material, though different depending on the kind of the materials and the like, is contained in a coating liquid for patterning substrate normally in the range of 0.01 to 95% by weight, and preferably in the range of 1 to 10% by weight. Thereby, a region that contains the cell adhesive material can be made a region excellent in the adhesiveness wi-*th cells.

3. Coating Liquid for Patterning Substrate

Next, a coating liquid for patterning substrate according to the present invention will be explained. The coating liquid for patterning substrate according to the present invention, as far as it contains the above-mentioned photocatalyst and the cell adhesive material, is not particularly restricted. As needs arise, it may appropriately contain a binder and the like.

In the present invention, it is particularly preferred to contain a binder. By containing the binder, when the coating liquid for patterning substrate is coated on, for instance, a base material, various characteristics such as that the coating can be easily applied and a formed layer can be imparted with the strength and the durability can be realized. Hereinafter, binders that are used in the present invention will be explained.

(Binder)

The binder used in the present invention, as far as it does not disturb the characteristics of the photocatalyst and the cell adhesive material, is not particularly restricted. Ones in which, for instance, the coating properties, the strength and durability when it forms a layer, and the like are tailored to the characteristics necessary for the coating liquid for patterning substrate can be used.

In the present invention, as the binder, a material that, at least after the energy irradiation, has the cell adhesion inhibiting properties that inhibits adhering to cells is preferably used. As such a material, one that has the cell adhesion inhibiting properties prior to the energy irradiation or one that becomes to have the cell adhesion inhibiting properties owing to an action of a photocatalyst in combination with energy irradiation may be used.

In the present invention, a material that becomes to have the cell adhesion inhibiting properties in particular owing to an action of a photocatalyst in combination with energy irradiation is preferably used as a binder. Thereby, in a region prior to the energy irradiation, the adhesiveness between the cell adhesive material and cells is not inhibited, and only a region where energy is irradiated can be lowered in the adhesiveness with the cells.

As materials that can be used as such a binder, for instance, ones in which a main skeleton has such a high bond energy that cannot be decomposed by the photo-excitation of the photocatalyst and an organic substituent can be decomposed by an action of the photocatalyst are preferably used. For instance, (1) organopolysiloxane that exhibits large strength by hydrolyzing or polycondensating chloro- or alkoxysilane or the like owing to a sol-gel reaction and the like, and (2) organopolysiloxane and the like in which reactive silicones excellent in the water repellency or oil repellency are crosslinked can be cited.

In the case of the (1), it is preferable to be organopolysiloxanes that are hydrolysis condensates or cohydrolysis condensates of at least one kind of silicon compounds expressed by a general formula:
YₙSiX₍₄₋ₙ₎
(Here, Y denotes an alkyl group, vinyl group, amino group, phenyl group or epoxy group, and X denotes an alkoxy group, acetyl group or halogen. n is an integer of 0 to 3.). The number of carbons of the group expressed with Y is preferably in the range of 1 to 20, and the alkoxy group shown with X is preferably a methoxy group, ethoxy group, propoxy group or butoxy group.

As the reactive silicone according to the (2), compounds having a skeleton expressed by a general formula below can be cited.

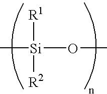

In the above general formula, n denotes an integer of 2 or more, R¹ and R² each represents a substituted or nonsubstituted alkyl group, alkenyl group, aryl group or cyanoalkyl group having 1 to 10 carbon, and a vinyl, phenyl and halogenated phenyl occupy 40% or less by mole ratio to a total mole. Furthermore, one in which R¹ and R² each is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material such as mentioned above is used, owing to an action of a photocatalyst in combination with energy irradiation, a surface of an energy-irradiated region can be made high in the hydrophilicity. Thereby, the adhesion with cells is inhibited, and one in which the cells do not adhere to a region where energy is irradiated can be obtained.

Together with the organopolysiloxanes, a stable organo silicium compound that does not cause a crosslinking reaction such as dimethylpolysiloxanes may be blended with a binder.

In the present invention, a decomposition substance or the like that causes such as a change in the wettability of a region where energy is irradiated to lower the adhesiveness with cells or that aides such a change may be contained.

As such decomposition substances, for instance, surfactants or the like that are decomposed and the like owing to an action of a catalyst in combination with energy irradiation to be hydrophilic and the like to result in lowering the adhesiveness with cells can be cited. Specifically, hydrocarbons of the respective series of NIKKO L BL, BC, BO, and BB manufactured by Nikko Chemicals Co., Ltd., and silicone base nonionic surfactants such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNE DS-401 and 402 manufactured by DAIKIN INDUSTRIES, Ltd., and Fluorad FC-170 and 176 manufactured by 3M can be cited, and cationic surfactants, anionic surfactants and amphoteric surfactants also can be used.

Other than the surfactants, oligomers and polymers such as polyvinyl alcohol; unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present invention, such a binder can be preferably contained, in the coating liquid for patterning substrate, in the range of 5 to 95% by weight, more preferably 40 to 90% by weight, and particularly preferably 60 to 80% by weight.

B. Patterning Substrate

A patterning substrate according to the present invention will be explained. The patterning substrate according to the present invention has a base material; and a cell culture patterning layer that is formed on the base material and has at least a photocatalyst and a cell adhesive material that has the adhesiveness with cells and can be decomposed or modified owing to an action of a photocatalyst in combination with energy irradiation.

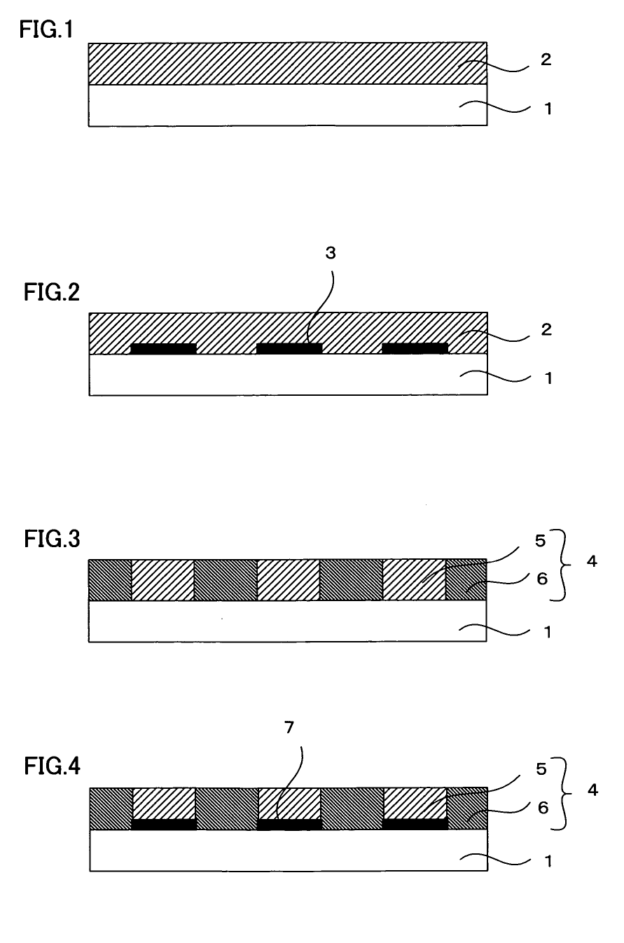

The patterning substrate according to the present invention, as shown in, for instance, FIG. 1, has a base material 1 and a cell culture patterning layer 2 formed on the base material. According to the present invention, since the cell culture patterning layer contains the photocatalyst and the cell adhesive material, when energy is irradiated on the cell culture patterning layer, the cell adhesive material can be decomposed or modified owing to an action of the photocatalyst and thereby can be rendered one to which the cells do not adhere. Furthermore, in a region where energy is not irradiated, the cell adhesive material remains; accordingly, the adhesiveness with the cells can be made excellent. Therefore, without requiring a special device or a complicated process, by irradiating energy in a pattern, a region having the cell adhesive material that is excellent in the adhesiveness with the cells and a region low in the adhesiveness with the cells can be easily formed.

In the patterning substrate according to the present invention, as shown in, for instance, FIG. 2, a light-shielding portion 3 may be formed on the base material 1. When the light-shielding portion is formed, in the case of energy being irradiated from a base material side, energy is not irradiated on the cell culture patterning layer on a region where the light-shielding portion is formed. Thereby, only the cell adhesive material on the region where the light-shielding portion is not formed can be decomposed or modified; accordingly, without using a photomask and the like, a region where the cells adhere and a region where the cells do not adhere can be readily formed.

Hereinafter, the respective configurations of the patterning substrate according to the present invention will be explained.

1. Cell Culture Patterning Layer

First, a cell culture patterning layer used in the present invention will be explained. The cell culture patterning layer used in the present invention, as far as it is formed on a base material described below and contains at least a photocatalyst and a cell adhesive material that can be decomposed or modified owing to an action of the photocatalyst in combination with energy irradiation, is not particularly restricted.

The cell culture patterning layer can be formed by coating, for instance, the above-mentioned coating liquid for patterning substrate to form a layer. In particular, the layer can be preferably formed with a coating liquid for patterning substrate that contains a binder of which adhesiveness with cells is lowered owing to an action of the photocatalyst in combination with energy irradiation. Thereby, when energy is irradiated on the cell culture patterning layer, the adhesiveness with cells in a region where energy is irradiated can be made lower, and only in a region where energy is not irradiated, the cells can be adhered with a highly precise pattern.

The coating liquid for patterning substrate and the like can be applied according to general methods. For instance, a spin coat method, spray coat method, dip coat method, roll coat method and bead coat method can be used.

The photocatalyst, cell adhesive material and binder contained in the cell culture patterning layer used in the present invention are identical to those described in the section of “A. Coating liquid for patterning substrate”. Accordingly, explanations thereof are not repeated.

A film thickness of the cell culture patterning layer, though properly selected depending on the kind and the like of the patterning substrate, is normally in the range of about 0.01 to 1.0 μm, and among these can be made in the range of about 0.1 to 0.3 μm.

2. Base Material

In the next place, a base material used in the present invention will be explained. The base material used in the present invention, as far as it is a layer on which the cell culture patterning layer can be formed, is not particularly restricted. For instance, inorganic materials such as metal, glass and silicium, and organic materials and the like typical in plastics can be used.

The flexibility and the like of the base material are properly selected according to the kind of the patterning substrate, applications or the like. Furthermore, the transparency of the base material is properly selected depending on such as the kind of the patterning substrate, or a direction in which energy that is irradiated to decompose or modify the cell adhesive material is irradiated. For instance, when the base material has such as the light-shielding portion and the energy is irradiated from a base material side and the like, the base material has the transparency.

On the base material according to the present invention, as mentioned above, a light-shielding portion may be formed. The light-shielding portion that can be used in the present invention, as far as it can shield energy that is irradiated on the patterning substrate, is not particularly restricted. For instance, a metal thin film that is made of chromium or the like and formed into a thickness of about 1000 to 2000 Å by a sputtering method, a vacuum deposition method or the like is formed and patterned to form a shielding portion. As the patterning method, an ordinary patterning method such as the sputtering can be used.

A method may be one by which a layer that contains light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds, and furthermore a photosensitive resin and an O/W emulsion type resin composition such as emulsified reactive silicone can be used. A thickness of such the resinous light-shielding portion can be set in the range of 0.5 to 10 μm. As a method of patterning such the resinous light-shielding portion, methods such as a photolithography method and a printing method that are generally used can be used.

The light-shielding portion may be formed on a surface of a side where the cell culture patterning layer is formed of the base material or may be formed on a surface of an opposite side.

In the case of forming the light-shielding portion, a primer layer may be formed between the cell culture patterning layer and the light-shielding portion. An action and function of the primer layer is not necessarily clear. However, it is considered that it has a function that when the primer layer is formed, impurities from the light-shielding portions and openings present between the light-shielding portions that become a factor that disturbs the decomposition or modification of the cell adhesive material in the cell culture patterning layer owing to an action of the photocatalyst, in particular, impurities such as residues generated when the light-shielding portions are patterned, metal and metal ion are inhibited from diffusing. Accordingly, when the primer layer is formed, the cell adhesive material can be decomposed or modified with high sensitivity, resulting in obtaining a high resolution pattern.

The primer layer in the present invention inhibits the impurities present not only in the light-shielding portion but also in the openings formed between the light-shielding portions from adversely affecting on an action of the photocatalyst; accordingly, the primer layer is preferably formed over an entire surface of the light-shielding portion including the openings.

The primer layer according to the present invention, as far as it is formed so that the light-shielding portion and the cell culture patterning layer may not be brought into contact, is not particularly restricted.

A material that forms the primer layer, though not particularly restricted, is preferably an inorganic material that is not likely to be decomposed owing to an action of the photocatalyst. Specifically, amorphous silica can be cited. When such amorphous silica is used, a precursor of the amorphous silica is preferably a silicon compound that is represented by a general formula, SiX₄, X being halogen, methoxy group, ethoxy group, acetyl group or the like, silanol that is a hydrolysate thereof, or polysiloxane having an average molecular weight of 3000 or less.

A film thickness of the primer layer is preferably in the range of 0.001 to 1 μm and particularly preferably in the range of 0.001 to 0.1 μm.

3. Patterning Substrate

Subsequently, a patterning substrate according to the present invention will be explained. The patterning substrate according to the present invention, as far as it is one in which a cell culture patterning layer is formed on the above-mentioned base material, is not particularly restricted. For instance, as needs arise, one in which another layer is further laminated and the like can be used.

C. Cell Culture Patterning Substrate A cell culture patterning substrate according to the present invention will be explained. The cell culture patterning substrate according to the present invention is a cell culture substrate having a base material and a cell culture patterning layer formed on the base material, the cell culture patterning layer having a cell adhesion portion that at least contains a photocatalyst and a cell adhesive material that has the adhesiveness with cells and can be decomposed or modified owing to an action of a photocatalyst in combination with energy irradiation; and a cell non-adhesion portion that at least contains a photocatalyst and where the cell adhesive material is decomposed or modified.

The cell culture patterning substrate according to the present invention, as shown in, for instance, FIG. 3, has a base material 1 and a cell culture pattern layer 4 formed on the base material 1, and the cell culture pattern layer 4 has a cell adhesion portion 5 and a cell non-adhesion portion 6.

According to the present invention, since the cell culture pattern layer has the cell adhesion portion and the cell non-adhesion portion, cells can be readily adhered only onto the cell adhesion portion, and even when cells are coated, for instance, over an entire surface of the cell culture pattern layer, only on the cell adhesion portion, cells can be adhered in high precision.

According to the present invention, after cells are adhered onto the cell adhesion portion, when energy is irradiated from, for instance, a base material side over an entire surface, a cell adhesive material of the cell adhesion portion thereto the cells are adhered can be decomposed or modified, and thereby one low in the adhesiveness with the cells can be formed. Thereby, the cells adhered onto the cell adhesion portion can be peeled and the cells formed in a pattern can be obtained. The energy irradiated at this time is one to an extent that does not affect on the cells.

In the present invention, as shown in, for instance, FIG. 4, in a pattern same as to the cell adhesion portion 5 of the cell culture pattern layer 4, a light-shielding portion 7 may be formed on the base material 1. When, by forming such light-shielding portion, the patterning substrate described, for instance, in the “B. Patterning substrate” is formed and energy is irradiated from a base material side, the cell adhesive material only on a region where the light-shielding portion is not formed can be decomposed or modified; that is, the cell non-adhesion portion can be readily formed.

When cells are adhered onto the cell adhesion portion of the cell culture patterning substrate according to the present invention followed by irradiating energy from the base material side, only onto the cell non-adhesion portion energy can be irradiated. Thereby, even when, such as the cells are adhered to the cell non-adhesion portion, owing to the energy irradiation, the cells on the cell non-adhesion portion can be removed and a highly precise pattern can be maintained.

Hereinafter, the respective configurations of the cell culture patterning substrate according to the present invention will be explained.

1. Cell Culture Pattern Layer

First, a cell culture pattern layer used in the cell culture patterning substrate according to the present invention will be explained. The cell culture pattern layer used in the present invention, as far as it is formed on a base material described below and has a cell adhesion portion and a cell non-adhesion portion, is not particularly restricted; however, it is preferable to contain a binder in the cell culture pattern layer. Thereby, various characteristics such as the strength and durability can be imparted to the cell culture pattern layer.

In the present invention, as the binder, in particular, a binder that is, owing to an action of a photocatalyst in combination with energy irradiation, altered in the wettability and the like to lower the adhesiveness with cells can be preferably contained. Thereby, since the cell non-adhesion portion can be rendered one that is lower in the adhesiveness with the cells, a cell culture pattern layer in which only on the cell adhesion portion cells can be adhered in highly precise pattern can be formed.

The cell adhesion portion is a region that contains a photocatalyst and a cell adhesive material that has the adhesiveness with cells and can be decomposed or modified owing to an action of the photocatalyst in combination with energy irradiation, and a region excellent in the adhesiveness with target cells. Here, having the adhesiveness with cells means to have the physicochemical cell adhesiveness or the biological cell adhesiveness such as explained in the section of “A. Coating liquid for patterning substrate”.

On the other hand, the cell non-adhesion portion is a region where at least a photocatalyst is contained, the cell adhesive material is decomposed or modified, and the adhesiveness with cells is low. Here, the cell adhesive material being decomposed or modified means that the cell adhesive material is not contained or in comparison with an amount of the cell adhesive material contained in the cell adhesion portion the cell adhesive material is less contained. For instance, when the cell adhesive material is one that is decomposed owing to an action of a photocatalyst in combination with energy irradiation, in the cell non-adhesion portion the cell adhesive material is not contained or a decomposition product and the like of the cell adhesive material is contained. Furthermore, when the cell adhesive material is one that is modified owing to an action of a photocatalyst in combination with energy irradiation, in the cell non-adhesion portion, a modified product thereof and the like is contained.

In the cell non-adhesion portion, as mentioned above, a binder low in the adhesiveness with cells is preferably contained. Thereby, the cells can be further inhibited from adhering onto the cell non-adhesion portion.

The cell adhesive material, photocatalyst, and binder are identical to those explained in the section of “A. Coating liquid for patterning substrate”; accordingly, explanations thereof are omitted. Hereinafter, a method of forming the cell culture pattern layer having the cell adhesion portion and the cell non-adhesion portion as mentioned above will be explained.

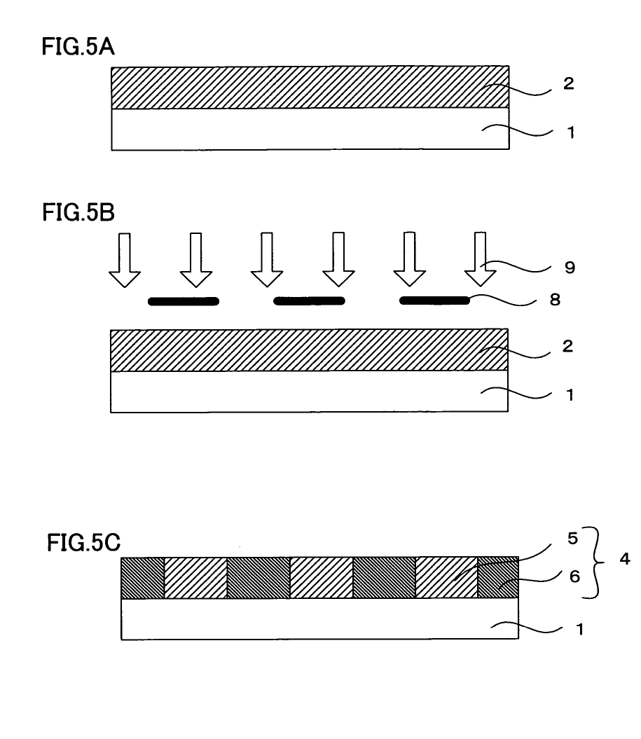

First, by using the coating liquid for patterning substrate explained in the “A. Coating liquid for patterning substrate” and the like, as shown in, for instance, FIGS. 5A to 5C, a cell culture patterning layer 2 that at least contains a photocatalyst and a cell adhesive material is formed on a base material 1 (FIG. 5A). Subsequently, energy 9 is irradiated on the cell culture patterning layer 2, in a pattern that forms the cell non-adhesion portion, by using, for instance, a photomask 8 and the like (FIG. 5B). Thereby, owing to an action of the photocatalyst contained in the cell culture patterning layer 2, the cell adhesive material in a region thereon energy is irradiated is decomposed or modified, and thereby a cell culture pattern layer 4 where the region where energy is irradiated becomes a cell non-adhesion portion 6 and a region where energy is not irradiated becomes a cell adhesion portion 5 is formed (FIG. 5C). The above-mentioned method of forming a cell culture patterning layer and the like are identical to the method explained in the “B. Patterning substrate”; accordingly, explanations thereof are not repeated. Furthermore, as mentioned above, when a binder of which adhesiveness with cells is lowered owing to an action of a photocatalyst in combination with energy irradiation is contained together with the photocatalyst and the cell adhesive material, owing to the energy irradiation, the adhesiveness of the binder with cells is also lowered, and thereby the adhesiveness with cells of the cell non-adhesion portion can be made further lower.

The energy irradiation (exposure) mentioned in the present invention is a concept that includes all energy line irradiation that can decompose or modify the cell adhesive material owing to an action of a photocatalyst in combination with energy irradiation, and is not restricted to visible light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which by use of the above-mentioned light source pattern irradiation is carried out through a photomask, a method of carrying out drawing irradiation in a pattern by use of laser such as excimer, YAG and the like can be applied. Furthermore, when the base material has a light-shielding portion in a pattern same as to that of the cell adhesion portion, energy can be irradiated over an entire surface from the base material side. In this case, there are advantages in that there are no needs of the photomask and the like and a process of positional alignment and the like.

An amount of irradiation of energy at the energy irradiation is an amount of irradiation necessary for decomposing or modifying the cell adhesive material owing to an action of a photocatalyst.

At this time, by irradiating energy with a layer that contains the photocatalyst heating, the sensitivity can be raised; accordingly, the cell adhesive material can be preferably and efficiently decomposed or modified. Specifically, it is preferable to heat in the range of 30 to 80° C.

The energy irradiation that is carried out through a photomask in the present invention, when the above-mentioned base material is transparent, may be carried out from either direction of a base material side or a side on which a cell culture pattern layer is formed. On the other hand, when the base material is opaque, it is necessary to apply energy irradiation from a side where the cell culture pattern layer is formed.

2. Base Material

A base material used for the cell culture patterning substrate according to the present invention will be explained. The base material used in the cell culture patterning substrate according to the present invention, as far as it can form the cell culture pattern layer, is not particularly restricted. For instance, one on which the light-shielding portion and the like is formed as mentioned above may be used.

As a base material, a light-shielding portion and the like that are used in the present invention, ones similar to those explained in the “B. Patterning substrate” can be used; accordingly, explanations thereof are not repeated here.

3. Cell Culture Patterning Substrate

A cell culture patterning substrate according to the present invention will be explained. A cell culture patterning substrate according to the present invention, as far as the above-mentioned cell culture pattern layer is formed on the base material, is not particularly restricted. As needs arise, the cell culture patterning substrate may be the one as such that another layer may be formed thereon.

D. Cell Culture Substrate

Next, a cell culture substrate according to the present invention will be explained. A cell culture substrate according to the present invention is one in which cells are adhered onto the cell adhesion portion in the above-mentioned cell culture patterning substrate.

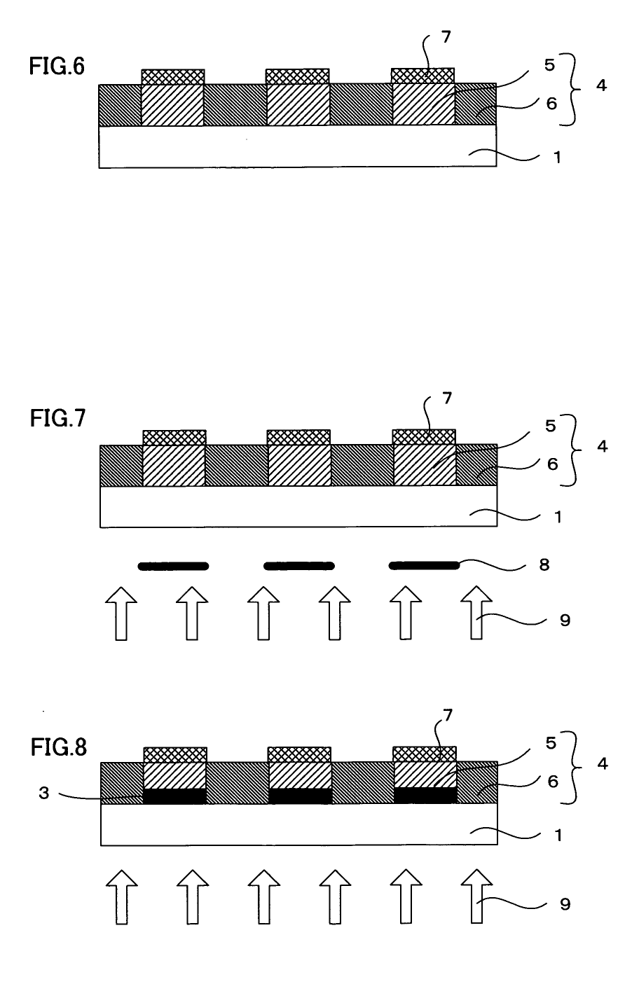

In the cell culture substrate according to the present invention, as shown in, for instance, FIG. 6, cells 7 are adhered only onto a cell adhesion portion 5 of the cell culture pattern layer 4, and, on a cell non-adhesion portion 6, cells 7 are not adhered.

According to the present invention, on the cell culture patterning substrate, a cell adhesion portion excellent in the adhesiveness with cells and a cell non-adhesion portion that does not have the adhesiveness with cells are formed. Accordingly, for instance, even when cells are coated over an entire surface of the cell culture patterning substrate, the cells can be adhered to the cell adhesion portion only and the cells on the cell non-adhesion portion can be readily removed. Thereby, without invoking a complicated process or using a processing liquid and the like that adversely affects on the cells, the cell culture substrate can be readily formed.

According to the present invention, when energy is irradiated over an entire surface of the cell culture substrate, a cell adhesive material of the cell adhesion portion thereto the cells are adhered can be decomposed or modified, resulting in forming one low in the adhesiveness with cells. Thereby, the cells and the like adhered onto the cell adhesion portion can be easily peeled, resulting in obtaining only those cells formed in a pattern. The energy irradiated at this time is energy to an extent that does not affect on the cells.

As mentioned above, in the case of a light-shielding portion being formed on a base material in a pattern same as to the cell adhesion portion, when energy is irradiated over an entire surface from the base material side as needs arise, cells adhered onto a cell non-adhesion portion can be removed; accordingly, a pattern in which cells are adhered to the cell adhesion portion can be maintained in high precision.

In the case of the above-mentioned light-shielding portion being not formed, when energy is irradiated by using a photomask or the like in which an opening is formed in a pattern same as to the cell non-adhesion portion, a highly precise pattern can be maintained.

Hereinafter, cells that are used in a cell culture substrate according to the present invention will be explained. Since an explanation of a cell culture patterning substrate is same as that in the “C. Cell culture patterning substrate”, here it is omitted.

(Cell)

As a cell used in a cell culture substrate according to the present invention, as far as it can adhere onto a cell adhesion portion of the cell culture patterning substrate but does not adhere to a cell non-adhesion portion, there is no particular restriction.

As a cell used in the present invention, except for, for instance, non-adhesive cells such as nervous tissue, liver, kidney, pancreas, blood vessel, brain, cartilage and blood corpuscle, all tissues present in an organism and cells derived therefrom can be used. Furthermore, since even for generally non-adhesive cells, recently, in order to adhere and fix, a technology of modifying a cell membrane is devised; accordingly, as needs arise, the non-adhesive cells, when this technology is applied, can be used in the present invention.

The respective tissues such as mentioned above are formed of cells having various functions; accordingly, it is necessary to select desired cells to use. For instance, in the case of the liver, it is formed of, other than hepatocytes, epithelial cells, endothelial cells, Kupffer's cells, fibroblasts, and fat-storing cells and the like. In this case, since the adhesiveness with a cell adhesive material is different depending on the kinds of the cells, in accordance with a cell strain, a cell adhesive material used in the cell adhesion portion and a composition ratio thereof have to be selected.

A method of adhering cells to the cell adhesion portion, as far as it can adhere cells only on a cell adhesion portion of the cell culture patterning substrate that has the cell adhesion portion and the cell non-adhesion portion, is not particularly restricted. For instance, cells may be adhered by use of an ink jet printer, a manipulator or the like; however, a method in which after a cell suspension is disseminated to adhere cells on the cell adhesion portion, unnecessary cells on a cell non-adhesion portion are washed with a phosphate buffer to remove the cells is generally used. As such a method, a method described in, for instance, a reference literature, Kevin E. Healy et al., “Spatial distribution of mammalian cells dicated by material surface chemistry”, Biotech. Bioeng. (1994), p. 792 can be used.

E. Method of Manufacturing Cell Culture Substrate

A method of manufacturing a cell culture substrate according to the present invention will be explained. A method of manufacturing a cell culture substrate according to the present invention is a method of manufacturing a cell culture substrate having a base material; a cell culture pattern layer that is formed on the base material and has a cell adhesion portion that at least contains a photocatalyst and a cell adhesive material that has the adhesiveness with cells and can be decomposed or modified owing to an action of the photocatalyst in combination with energy irradiation and a cell non-adhesion portion that at least contains a photocatalyst and where the cell adhesive material is decomposed or modified; and cells adhered onto the cell adhesion portion, the method comprising:
a cell maintaining process for irradiating energy on the cell non-adhesion portion to maintain a pattern of the cell adhered onto the cell adhesion portion, after a cell adhering process for adhering a cell on the cell adhesion portion is carried out.

A method of manufacturing a cell culture substrate according to the present invention is, as shown in, for instance, FIG. 7, a method of manufacturing a cell culture substrate having a base material 1; a cell culture pattern layer 4 that is formed on the base material 1 and has a cell adhesion portion 5 and a cell non-adhesion portion 6; and cells 7 formed on the cell adhesion portion 5. In the present invention, a cell maintaining process, which irradiates energy 9 on the cell non-adhesion portion 6 by using, for instance, a photomask 8 and the like to maintain a pattern of cells 7 adhered onto the cell adhesion portion 5, is carried out after a cell adhering process for adhering-cells 7 on the cell adhesion portion 5 is carried out. Thereby, even when the cells adhere onto the cell non-adhesion portion, owing to an action of a photocatalyst contained in the cell non-adhesion portion, proteins, organics, cells and the like on the cell non-adhesion portion can be removed; accordingly, a cell culture substrate only on the cell adhesion portion of which cells are adhered in high precisison can be obtained.

Hereinafter, the cell maintaining process in the method of manufacturing a cell culture substrate according to the present invention will be explained.

(Cell Maintaining Process)

The cell maintaining process in the method of manufacturing a cell culture substrate according to the present invention is the process in which after the cell adhering process for adhering cells onto the cell adhesion portion is carried out, energy is irradiated on the cell adhesion portion to maintain a pattern of cells adhered on the cell adhesion portion, and as far as it can allow maintaining a pattern of cells on the cell adhesion portion by irradiating energy on the cell non-adhesion portion, there is no particular restriction on a method of irradiating energy and the like.

In the present invention, as shown in, for instance, FIG. 7, a method and the like in which by using photomask 8 and the like having an opening in a pattern same as to the cell non-adhesion portion energy 9 is irradiated can be cited. Furthermore, as shown in, for instance, FIG. 8, when a light-shielding portion 3 on a base material 1 is formed in a pattern same as to the cell adhesion portion 5, a method in which energy is irradiated over an entire surface from the base material 1 side to irradiate energy 9 only on the cell non-adhesion portion 6 can be used.

At that time, energy that is irradiated, as far as it can remove the cells adhered on the cell non-adhesion portion owing to an action of the photocatalyst in combination with energy irradiation, is not particularly restricted. Specifically, it can be same as that explained in the section of the cell culture pattern layer of the “C. Cell culture patterning substrate”; accordingly, explanation thereof is not repeated here.

As for a timing when the process is applied, it may be carried out immediately after the adhering cells where cells are adhered on the cell adhesion portion is carried out, or when cells are cultured for a predetermined period on the cell adhesion portion, in order to avoid inconveniences such as that the cells adhere on the cell non-adhesion portion to result in a wide pattern and the like, in accordance with a kind and state of the cells, the timing may be properly selected. The process, also, may be repeated.

The cell adhering process for adhering cells onto the cell adhesion portion is same as that of the method of adhering cells explained in the section of the “D. Cell culture substrate”, therefore, an explanation here is omitted.

(Others)

In a method of manufacturing a cell culture substrate according to the present invention, other than the energy irradiating process and the cell adhering process mentioned above, for instance, a process in which a coating liquid for patterning substrate containing a photocatalyst and a cell adhesive material is coated on a base material to form a cell culture patterning layer, and a process in which energy is irradiated in a pattern on the cell culture patterning layer to form a cell culture pattern layer having a cell adhesion portion and a cell non-adhesion portion can be included as needs arise.

A photocatalyst, cell adhesive material, base material, cells and the like that are used in the method of manufacturing a cell culture substrate according to the present invention and a method of forming the cell culture pattern layer are same as that described in the section of the “D. Cell culture substrate”, explanations thereof are not repeated here.

The present invention is not restricted to the above-mentioned embodiments. The above embodiments are only for exemplification, and all that have a configuration substantially same as a technical idea described in the range of claims of the present invention and exhibit identical operations and effects are included in a technical range of the present invention.

### EXAMPLES

Hereinafter, examples are shown and thereby the present invention will be more specifically described.

### Example 1

(Preparation of Cell Culture Patterning Substrate)

Blended were 3 g of isopropyl alcohol, 0.4 g of organosilane (trade name: TSL8114, manufactured by GE Toshiba Silicones), 0.04 g of N-(2-aminoethyl)-3-aminopropyl trimethoxysilane (manufactured by Huels America Inc.) and 1.5 g of photocatalyst inorganic coating agent (trade name; ST-K01, manufactured by ISHIHARA SANGYO KAISHA, LTD.), followed by heating at 100° C. for 20 min while stirring.

This solution was coated by a spin coating method on a quartz glass substrate processed alkali treatment beforehand, the substrate was heated at 150° C. for 10 min to dry, followed by forwarding a hydrolysis and a polycondensation reaction, and thereby a photocatalyst-containing cell adhesive material film substrate in which the photocatalyst is solidly fixed in organo-polysiloxane and that has a film thickness of 0.2 μm was obtained.

On the substrate, by use of a photomask, UV exposure was carried out for 900 sec at an illuminance of 300 mW/cm² with a mercury lamp (wavelength: 365 nm), and thereby a cell culture substrate in which a non-exposed portion is a cell adhesion portion and an exposed portion is a cell non-adhesion portion was obtained.

(Adhering Cells)

A procedure and the like of the cell culture was, based on a method described in, for instance, Asakura Shoten “Soshikibaiyo no Gijyutsu, Dai SanHan, Kiso”, the Japanese Tissue Culture Association (editer), carried out as follows.

A liver removed from a rat was transferred in a petri dish and chopped into 5 mm size with a surgical knife, followed by adding 20 ml of DMEM culture media and lightly suspending with a pipette, further followed by filtrating with a cell filter. An obtained coarse cell dispersed suspension liquid was centrifuged at 500 to 600 rpm for 90 s, followed by suctioning a supernatant to remove. To remaining cells, a DMEM culture media was added anew, followed by centrifuging again. This operation was repeated three times, and thereby substantially homogeneous hepatocytes were obtained. To the obtained hepatocytes, 20 ml of the DMEM culture media was added to suspend, and thereby a hepatocyte suspension was prepared.

To 14.12 g of Waymouth MB752/1 culture media (containing L-glutamine and lacking NaHCO₃) (manufactured by GIBCO), 900 ml of distilled water was added. Thereto, 2.24 g of NaHCO₃, 10 ml of amphotericin B (ICN) and 10 ml of penicillin streptomycin liquid (manufactured by GIBCO) were added, followed by agitating. After this solution was controlled to pH 7.4, a total amount was adjusted to 1000 ml, followed by filtrating and sterilizing with a membrane filter of 0.22 μm, and thereby a Waymouth MB752/1 culture media liquid was prepared.

The previously prepared hepatocyte suspension was suspended in the previously prepared Waymouth MB752/1 culture media liquid, followed by disseminating on the above-mentioned cell culture patterning substrate having a cell adhesion portion and a cell non-adhesion portion. The substrate was stood still in a 5% CO₂-gassed incubator at 37° C. for 24 hr to adhere hepatocytes over an entire surface of the substrate. The substrate was washed twice with PBS to remove non-adhesion cells and died cells, followed by replacing by a new culture media liquid.

While replacing the culture media liquid, the cells were cultured on up to 48 hr, followed by observing cells by an optical microscope, and it was confirmed that cells adhered on the cell culture patterning substrate along a cell adhesion portion.

### Example 2

Except that instead of a quartz substrate used in example 1, a quartz substrate provided thereon with a striped light-shielding layer having a light-shielding portion of 80 μm and a space portion of 300 μm was used and, without using a photomask and the like, UV light was irradiated from a back surface side of the substrate, similarly to example 1, a cell culture patterning substrate was prepared and cells were cultured.

In this case too, when the cells were observed by an optical microscope, it was confirmed that cells adhered on the cell culture patterning substrate along a cell adhesion portion.

1. A patterning substrate, comprising:
a base material; and
a cell culture patterning layer that is formed on the base material and contains at least a photocatalyst and a cell adhesive material that has an adhesiveness with a cell and is decomposed or modified owing to an action of a photocatalyst in combination with energy irradiation.

2. The patterning substrate according to claim 1, wherein a light-shielding portion is formed in a pattern on the base material.

3. The patterning substrate according to claim 1, wherein the cell culture patterning layer contains a binder.

4. The patterning substrate according to claim 2, wherein the cell culture patterning layer contains a binder.

5. A cell culture patterning substrate comprising:
a base material; and
a cell culture pattern layer formed on the base material,
wherein the cell culture pattern layer has a cell adhesion portion that contains at least a photocatalyst and a cell adhesive material that has an adhesiveness with a cell and is decomposed or modified by an action of the photocatalyst in combination with energy irradiation; and a cell non-adhesion portion that contains at least a photocatalyst and where the cell adhesive material is decomposed or modified.

6. The cell culture patterning substrate according to claim 5, wherein a light-shielding portion is formed in the same pattern to the cell adhesion portion on the base material.

7. The cell culture patterning substrate according to claim 5, wherein the cell culture patterning layer contains a binder.

8. The cell culture patterning substrate according to claim 6, wherein the cell culture patterning layer contains a binder.

9. A cell culture substrate, wherein a cell is adhered onto the cell adhesion portion in the cell culture patterning substrate according to claim 5.

10. A cell culture substrate, wherein a cell is adhered onto the cell adhesion portion in the cell culture patterning substrate according to claim 6.

11. A cell culture substrate, wherein a cell is adhered onto the cell adhesion portion in the cell culture patterning substrate according to claim 7.

12. A cell culture substrate, wherein a cell is adhered onto the cell adhesion portion in the cell culture patterning substrate according to claim 8.

13. A coating liquid for patterning substrate comprising:
at least a photocatalyst; and
a cell adhesive material that has an adhesiveness with a cell and is decomposed or modified owing to an action of the photocatalyst in combination with energy irradiation.

14. The coating liquid for patterning substrate according to claim 13, wherein the coating liquid contains a binder.

15. A method of manufacturing a cell culture substrate having a base material; a cell culture pattern layer that is formed on the base material and has a cell adhesion portion that contains at least a photocatalyst and a cell adhesive material that has an adhesiveness with a cell and is decomposed or modified owing to an action of the photocatalyst in combination with energy irradiation and a cell non-adhesion portion that contains at least a photocatalyst and where the cell adhesive material is decomposed or modified; and a cell adhered onto the cell adhesion portion, the method comprising
a cell maintaining process for irradiating energy on the cell non-adhesion portion to maintain a pattern of the cell adhered onto the cell adhesion portion, after a cell adhering process for adhering a cell on the cell adhesion portion is carried out.
